(19) United States
(12) Patent Application Publication
(10) Pub. No.: US 2024/0161893 A1
(43) Pub. Date: May 16, 2024

(54) DETERMINING A THERAPY EFFICACY

(71) Applicant: EosDx Inc., Mountain View, CA (US)

(72) Inventors: Alexander P. Lazarev, Lake Forest, CA (US); Delvin Tai Wai YUK, Atherton, CA (US); Pavel Lazarev, Box Elder, SD (US)

(73) Assignee: EosDx Inc., Mountain View, CA (US)

(21) Appl. No.: 18/056,219

(22) Filed: Nov. 16, 2022

(51) Int. Cl. G16H 20/10; A61B 5/00

(52) U.S. Cl. CPC G16H 20/10; A61B 5/0075

(57) ABSTRACT

The present disclosure relates to determining a therapy efficacy. A method for determining an efficacy of a therapy for a disease in a human patient can include measuring a molecular structure of a biological tissue of a patient at a first time and at a second time using a non-invasive biological tissue characterization technique. The method can further include observing a change of the molecular structure of the biological tissue between the first time and the second time, and determining the efficacy of the therapy based on the observed change in the molecular structure of the biological tissue. Before the first time, or between the first time and the second time, the patient received the therapy.

### BACKGROUND

Determining the efficacy of therapies for various diseases in human patients can be challenging due to the limited pharmacokinetic (PK) and/or pharmacodynamic (PD) information available. Furthermore, identifying criteria for determining the efficacy of different kinds of therapies, such as those with different treatment protocols, purposes of a therapeutic agent used, dose selections, and/or dosing intervals, can also be challenging using conventional methods. Therapy efficacy can furthermore be influenced by a pharmaceutical formulation of a therapeutic agent used in the therapy. For example, clinically relevant differences have been reported in some compounded medications compared with corresponding approved formulations. In some cases, clinicians must, therefore, critically weigh the use of compounded drugs, especially if there is a concern of therapeutic failure or an adverse event.

Determining a therapy efficacy has been done using pharmacometric methods, which have hugely benefited from progress in analytical and computer sciences during the past decades, and play nowadays a central role in the clinical development of new medicinal drugs. These methods have also been translated into patient care through therapeutic drug monitoring (TDM). For example, TDM approaches have been used with the drug imatinib, which is a targeted anticancer agent.

The suboptimal use of antimicrobial drugs in therapies has also been shown to contribute to increasing antimicrobial resistance and may lead to therapy failure at the individual patient level. Considering the significant pharmacokinetic (PK) variation in special patient populations and the variability in pathogen susceptibility, personalized dosing of antimicrobial drugs is known to be important. For example, dosing based on PK and PD properties of antibiotics guided by TDM can be used to improve the efficacy and reduce the toxicity for the individual patient. Model-informed precision dosing (MIPD) has also been used to provide a means of predicting drug response and dose requirements in individual patients based on individual patient and pathogen characteristics.

It is difficult to develop therapies for some diseases. For example, nontuberculous mycobacteria can cause minimally symptomatic self-limiting infections to progressive and life-threatening disease of multiple organs. Guidelines have been published to guide therapy for this emerging infectious disease, with limited success due to the complexity of therapy, the potential for acquired resistance, the toxicity of treatment, and a high treatment failure rate. Given the long duration of therapy, complex multi-drug treatment regimens, and the risk of drug toxicity, TDM methods have also been used to optimize treatment.

Non-invasive biological tissue characterization techniques, for example low angle fiber X-ray diffraction techniques, have been used to measure tissue samples in humans to identify different types of cancer. Fiber diffraction patterns of skin or fingernails, using X-ray sources, have been used as a biometric diagnostic method. The results obtained produce characteristic diffraction patterns which are distinctive and reproducible for a number of cancers including breast cancer, prostate cancer, colon cancer and melanoma.

### SUMMARY

The present disclosure relates to determining a therapy efficacy. In some embodiments, a method for determining an efficacy of a therapy for a disease in a human patient includes: measuring a molecular structure of a biological tissue of a patient at a first time and at a second time using a non-invasive biological tissue characterization technique; observing a change of the molecular structure of the biological tissue between the first time and the second time; and determining the efficacy of the therapy based on the observed change in the molecular structure of the biological tissue, wherein before the first time or between the first time and the second time the patient received the therapy.

### BRIEF DESCRIPTION OF THE DRAWINGS

FIG. 1 is a flowchart of an example of a method for determining an efficacy of a therapy for a disease in a human patient, in accordance with some embodiments.

FIG. 2 is a flowchart of an example of a method for determining an efficacy of a therapy for a disease in a human patient, in accordance with some embodiments.

FIGS. 3A and 3B show examples of X-ray tissue diffractometers, in accordance with some embodiments.

FIG. 4 is a flowchart of an example of a method for determining an efficacy of a therapy for a disease in a human patient, in accordance with some embodiments.

### DEFINITIONS

As used herein, the term “efficacy” generally refers to the capacity to produce an effect (e.g, lower blood pressure, or change the molecular structure of a biological tissue from a structure indicative of a diseased state to a structure indicative of a healthy state). Efficacy can conventionally be assessed accurately in some situations, such as when patients are selected by proper criteria and strictly adhere to the dosing schedule. For example, conventionally, efficacy can be measured under expert supervision in a group of patients most likely to have a response to a drug, such as in a controlled clinical trial.

As used herein, the term “precision medicine” generally refers to a medical model that proposes the customization of healthcare, with medical decisions, treatments, practices, or products being tailored to a subgroup of patients, instead of a one-drug-fits-all model. In precision medicine, diagnostic testing is often employed for selecting appropriate and optimal therapies based on the context of a patient's genetic content or other molecular or cellular analysis.

As used herein, the term “metabolism” generally refers to any chemical process occurring within or between cells. There are two types of metabolism: anabolism, where smaller molecules are synthesized to make larger ones; and catabolism, where larger molecules are broken down into smaller ones. Metabolism is an umbrella term referring to any cellular process that involves a chemical reaction. Glycolysis is an example of a catabolic cellular process; in this process, glucose is broken down into pyruvate.

As used herein, the term “enzyme” generally refers to catalyst which is required to start most chemical reactions within cells. Enzymes, which are large protein molecules found in the body, can be excellent catalysts because they can change the chemicals within the cells without changing themselves.

As used herein, the term “polymorphisms” generally refers to different forms of a DNA sequence. “Poly” means many, and “morph” means form. Polymorphisms are a type of genetic diversity within a population's gene pool. They can be used to map (locate) genes such as those causing a disease, and they can help match two samples of DNA to determine if they come from the same source. Depending on its exact nature, a polymorphism may or may not affect biological function. For example, polymorphism of metabolic enzyme genes may be related with risk of prostate cancer.

As used herein, the term “gut microbiota” generally refers to microorganisms including bacteria and archaea, that live in the digestive tracts of vertebrates including humans, and of insects. The gastrointestinal metagenome (sometimes defined as the microbiome) is the aggregate of all the genomes of gut microbiota. In the human, the gut is the main location of human microbiota. The gut microbiota has broad impacts, including effects on colonization, resistance to pathogens, maintaining the intestinal epithelium, metabolizing dietary and pharmaceutical compounds, controlling immune function, and even behavior through the gut-brain axis.

As used herein, the term “non-invasive observation” or “non-invasive biological tissue characterization” of a patient refers an observation or characterization technique that does not include the introduction of instruments into the body of a patient. For example, non-invasive observation or characterization can exclude blood sampling and the introduction of pathogenic viruses and bacteria into the body. Non-invasive observation or characterization can advantageously spare the patient from pain. In some cases, non-invasive observation or characterization can eliminate radiation exposure to the body.

As used herein, the term “biological tissue” or “biological tissue sample” generally refers to tissue (or tissue samples) of a patient. For example, biological tissue can include materials of living organs that contain structural molecular components and functional components like cells, muscles, and skin, as well as detachable structures like nails and hairs. In some cases, biological tissue can contain biological molecular structures such as collagens, keratins and glycoproteins that diffract X-ray light.

As used herein, the term “X-ray tissue diffractometer” generally refers to a diffractometer configured to record diffraction data from one or more tissue sites including structural and functional molecular structures, for example sites in cells, skin and hair.

As used herein, the term “cancer” generally refers to a proliferative disorder caused or characterized by a proliferation of cells which have lost susceptibility to normal growth control. Cancers of the same tissue type usually originate in the same tissue and may be divided into different subtypes based on their biological characteristics. In some cases, malignant tumors (cancer) can be further classified according to morphological characteristics, such as: a) epithelial (papillomas, adenomas, carcinomas, cysts, dermatomas); b) connective tissue (fibroids, myxomas, lipomas, chondromas, osteomas, melanosarcomas); c) nervous tissue (gliomas, neurinomas, meningiomas); d) muscle (fibroids, rhabdomyomas); e) vascular (hemangiomas and lymphangiomas); f) mixed (osteosarcomas and fibromyxochondroma, fibrochondroosteoma).

As used herein, the term “pharmacokinetic (PK) data” generally refers to reports including data about how a patient's body (or tissue) responds to a drug, including absorption to metabolism, distribution, and excretion.

As used herein, the term “pharmacodynamic (PD) studies” generally refers to studies that describe how a drug interacts with the body (or tissue) of a patient in a dose dependent manner, for example, including anticipated and unanticipated (adverse) responses.

As used herein, the term “image segmentation” generally refers to a method used in digital image processing and computer vision, including a process of partitioning a digital image into multiple image segments (also known as image regions, image objects, or sets of pixels). The goal of segmentation can be to simplify and/or change the representation of an image into something that is more meaningful and easier to analyze. Image segmentation is typically used to locate objects and boundaries (e.g., lines, curves, etc.) in images. Additionally, image segmentation can include the process of assigning a label to every pixel in an image such that pixels with the same label share certain characteristics.

As used herein, the term “recognition of objects” or “object recognition” generally refers to a field of computer vision wherein objects are found and/or identified in an image (or one or more images from a video sequence).

### DETAILED DESCRIPTION

The present disclosure provides a method for determining an efficacy of a therapy (e.g., a drug treatment, or other clinical treatment) including observing changes in a molecular structure of biological tissue samples over time. In some cases, the biological tissue can be collagens, keratins, or glycoproteins that diffract X-rays. In some cases, the measured data can be analyzed using digital image processing, for example including pattern recognition and the recognition of objects in X-ray diffraction images.

X-ray diffraction systems and methods for characterizing biological tissue samples are further described in U.S. patent application Ser. Nos. 17/593,846 and 17/448,888, which are hereby incorporated by reference in their entireties.

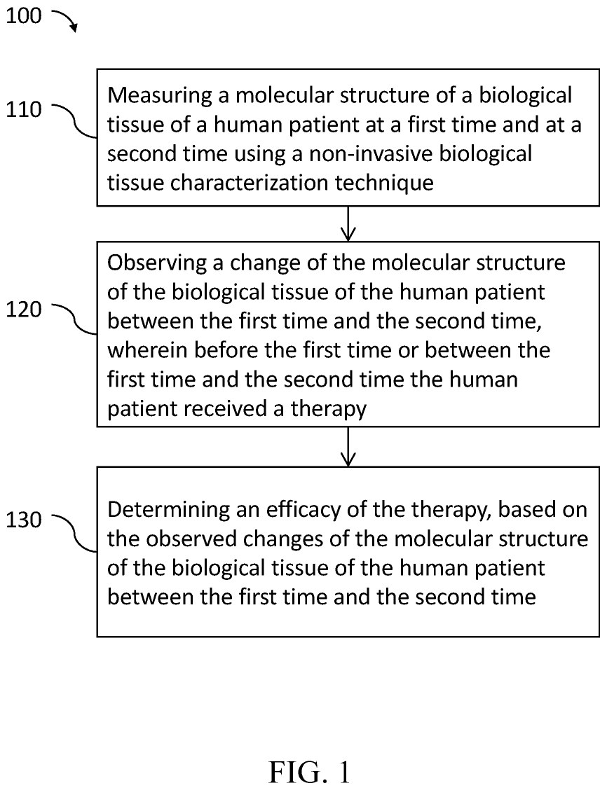

FIG. 1 is a flowchart of an example of a method 100 for determining an efficacy of a therapy for a disease in a human patient including the following blocks. In block 110, a molecular structure of a biological tissue of a patient is measured at a first time and at a second time using a non-invasive biological tissue characterization technique. At block 120, a change of the molecular structure of the biological tissue is observed (or determined, or calculated) between the first time and the second time, such as by analyzing data from the measurements from non-invasive biological tissue characterization technique at the first time and the second time using a computer processor. In block 130, the efficacy of the therapy is determined based on the observed change in the molecular structure of the biological tissue. In some cases, an observed change in the molecular structure of a biological tissue can be that no change (or no significant change) occurred in the molecular structure of a biological tissue. In this case, the patient received the therapy either before the first time, or between the first time and the second time, thereby allowing the observed change in the molecular structure of the biological tissue to provide feedback regarding the efficacy of the therapy.

Therapies for diseases such as cancer (e.g., melanoma, breast, colon, and prostate cancers) and Alzheimer's disease can change structural properties (e.g., an alignment of molecules) of a biological tissue, which can be monitored using characterization techniques (e.g., X-ray diffraction), and the efficacy of such therapies can be determined using the methods described herein (e.g., method 100). Therapies for other diseases, the efficacy of which can be evaluated using the current methods, include therapies for diseases of the immune system, diseases of the skin, oncological disease, rheumatic diseases, urological diseases, endocrine diseases, diseases of veins and lymph nodes, diseases of the mammary glands, diseases of respiratory organs, diseases of digestive organs, diseases of heart and blood vessels, diseases of the colon, diseases of the ear, diseases of the throat, and diseases of the nose. For example, an oncological disease can include cancer of the stomach, liver, rectum and colon, esophagus, pancreas, bladder, vagina, lung, oropharynx, nasopharynx, oral mucosa, tongue, skin, brain, thyroid, prostate, breast, cervix, and/or ovary. Therapy efficacy (e.g., the efficacy of an administered drug) can be determined by characterizing the changes in the structural properties of a biological tissue of a patient (e.g., a sample of biological tissue taken from the patient measured ex vivo, or a biological tissue of the patient measured in vivo) over the course of treatment. In some cases, changes of a structure of an extracellular matrix on an organism level in response to morphogenesis in organs allows monitoring of the changes in the extracellular matrix in sites remote from site of morphogenesis. For example, a molecular structure of a biological tissue that is remote from a site of a disease, such as hair or nails, can be measured to determine the efficacy of a treatment for the disease. In some cases, an effective therapy that causes diseased tissue to become healthy (or be eliminated) can be confirmed by directly monitoring improvements in the structural properties of the biological tissue of the patient (e.g., in measurements taken in the same region of the patient over time, where the tissue in that region may or may not change). Conversely, an ineffective therapy can be identified by directly observing that diseased tissue remains (e.g., in measurements taken of biological tissue in (or from) the same location of the patient) throughout (and after) the course of treatment.

In some embodiments, the methods disclosed herein are precision medicine methods and provide rational dosing regimens adapted to patients' characteristics that can relevantly benefit patients in clinical practice, for example, by preventing or correcting both under-dosing deleterious to therapeutic efficacy and overdosing leading to toxicity and subsequent treatment cessation.

In some embodiments, method 100 of FIG. 1 further includes adapting a dosing regimen of a therapeutic agent that is used in the therapy to characteristics of the human patient, wherein the dosing regimen prevents or corrects both under-dosing deleterious to the efficacy of the therapy and overdosing leading to toxicity.

In some embodiments, the methods disclosed herein can be used to determine the efficacy of a therapy that uses one or more of the following therapeutic agents: anticancer drugs, biologics, antiretrovirals, antiinfectives, and psychotropic agents. “Biologics” can include any agent(s) manufactured in a living system (e.g., in a microorganism, or a plant or animal cell using recombinant DNA technology). In some cases, biologics are large complex molecules, or mixtures of molecules.

In some embodiments, the methods disclosed herein include optimizing the efficacy of the therapy for a specific patient, including taking into account one or more characteristics of: a gender of the patient, a metabolic enzyme polymorphism of the patient, a gut microbiota of the patient, a time of administration of a first therapeutic agent that is used in the therapy, a presence of hepatic or renal disease in the patient, and an interaction between the first therapeutic agent and a second therapeutic agent used by the patient.

In some embodiments, the methods disclosed herein include measuring (e.g., in block 110 of method 100 in FIG. 1) a molecular structure of biological tissue that diffracts X-ray light, such as a collagen, a keratin, and/or a glycoprotein.

In some embodiments, the biological tissue measured in the methods disclosed herein (e.g., in block 110 of method 100 in FIG. 1) include breast tissue, brain tissue, hair, nail, and/or skin.

In some embodiments, the non-invasive biological tissue characterization technique of the methods disclosed herein includes one or more of: X-ray diffraction (e.g., using a tissue diffractometer comprising a two-dimensional pixel detector), luminescent spectroscopy, selective laser spectroscopy, Raman spectroscopy, spectroscopy in the visible spectral region (e.g., 400-740 nm), and infrared spectroscopy.

In some embodiments, the methods disclosed herein (e.g., in block 120 of method 100 in FIG. 1) the non-invasive observation of the changes in the molecular structure of the biological tissue samples is implemented by a device such as X-ray tissue diffractometer comprising a two-dimensional pixel detector, luminescent spectroscope, selective laser spectroscope, Raman spectroscope, spectroscope in the visible spectral region (400-740 nm), and infrared spectroscope. In some embodiments, one or more processors (or computers, or servers) are used to process data from the device. In some embodiments, the processed data can further be used by the one or more processors (or computers, or servers) to determine the efficacy of the treatment (e.g., in block 130 in method 100 in FIG. 1).

In some cases, in block 120 of method 100, the observed change in the molecular structure of the biological tissue can indicate a change in a trajectory. For example, a therapy can be started at a first time (i.e., a first point in time, such as a certain date), and cause a first observed change in the molecular structure of the biological tissue, and that can indicate a first efficacy of the therapy. Subsequently, a second change in the molecular structure of the biological tissue can be observed at a second time (i.e., a second point in time, such as a second date), which indicates a second efficacy of the therapy (e.g., in a second time period) that is different from the first efficacy. In some cases, the therapy can be more efficacious in the first observation, and in other cases, the therapy can be more efficacious in the second observation.

In some embodiments, the methods disclosed herein include measuring concentrations of a therapeutic agent (e.g., a drug) in the biological tissue samples until a defined target concentration is reached, for example, associated with optimal efficacy and minimal toxicity. In some cases, the method can include measuring a concentration of a drug in the biological tissue sample using an optical technique, such as luminescent spectroscope, selective laser spectroscope, Raman spectroscope, spectroscope in the visible spectral region (400-740 nm), and infrared spectroscope. The measurement of the concentrations of the therapeutic agent can improve patient care by providing information about the amount of the therapeutic agent in the biological tissue over time, which can be used to determine (or adapt) a dosing regimen of the therapeutic agent. For example, the dosing regimen can be adapted and the concentrations of the therapeutic agent in the biological tissue can be measured until the concentration of the therapeutic agent in the biological tissue reaches a predefined target concentration (e.g., associated with optimal efficacy and minimal toxicity).

In some embodiments, more than two measurements of a molecular structure of a biological tissue of a patient are taken over time in block 110 of method 100. In such cases, in block 120 changes in the molecular structure of the biological tissue can be observed using an algorithm to analyze the change in the molecular structure over time (wherein the change can include no change, or no significant change). For example, the algorithm can analyze the measurement data to determine the change in the molecular structure after each successive measurement. The output from the algorithm indicating the change in molecular structure over time can be used to determine the efficacy of the therapy, and/or change the therapy (e.g., change the dose or dosing interval of a therapeutic agent, or change to a different therapeutic agent). In cases where a series of measurements are taken, the “first time” in method 100 can correspond to the first measurement in the series of measurements (either before or after starting the therapy), or the “first time” in method 100 can correspond to a measurement other than the first measurement in the series of measurements. Additionally, when a series of measurements are taken, the first time and the second time can be consecutive or non-consecutive measurements.

In block 110 of method 100, measurements of the molecular structure of the biological tissue can be taken at various times following the initiation of, during treatment by, or after completion of a therapy. The data from the measurements can be analyzed after each successive measurement or at selected intervals using a multi-step algorithm. In some cases, the multi-step algorithm includes statistical analyses to determine the change of the molecular structure of the biological tissue in block 120 (e.g., after each measurement). In some cases, more than one measurement can be taken at a particular time (e.g., on the same day, or in a single measurement session), for example, to provide more data for statistical analyses. In some cases, the statistical analyses can include fitting measured data to a function (e.g., a linear function, a polynomial function, an exponential function, or a logarithmic function) to determine the change of the molecular structure of the biological tissue. In this process, regression coefficients of the fitted functions can be determined using the statistical analyses. In some cases, comparison of regression coefficients of functions that have been fit to the measured data using multiple measurements can improve the statistical significance of an observed change of the molecular structure of the biological tissue over time. In some cases, the statistical analyses may include a determination of a pair-wise distance distribution function, a determination of a Patterson function, a calculation of a Porod invariant, a Fourier transformation, a cluster analysis, a dispersion analysis, a determination of one or more molecular structural periodicities, or any combination thereof. In some cases, the multi-step algorithm can analyze the clustering of data (e.g., derived from the analysis of image data, diffraction data, subject data, or any combination thereof) and re-evaluate observed changes in sample data characteristics and clustering over time. In some cases, the multi-step algorithm can plot patient sample data points in an n-dimensional space defined by two or more parameters of the therapy (e.g., a degree of crystallinity of a molecular structure, time, a therapeutic agent dose, etc.) and analyze the data for changes in the molecular structure. For example, the distance or changes in distance between data points or clusters of data points may be calculated as a function of time. In some instances, the proximity of a new data point to the previous data point(s), or the trajectory of certain data clusters (or the gradient of the trajectory) can describe the observed change in the molecular structure of the biological tissue over time. These factors may be used as indicators for the efficacy of the therapy and/or can be interpreted by a physician in terms of therapeutic efficacy. In some instances, the output of the multi-step algorithm can be used to directly monitor the efficacy of a therapeutic treatment. Comparing the results of assessments for multiple patients can also provide indications of the efficacy of a therapy (e.g., the effectiveness of a therapeutic agent) in a patient or in groups of patients.

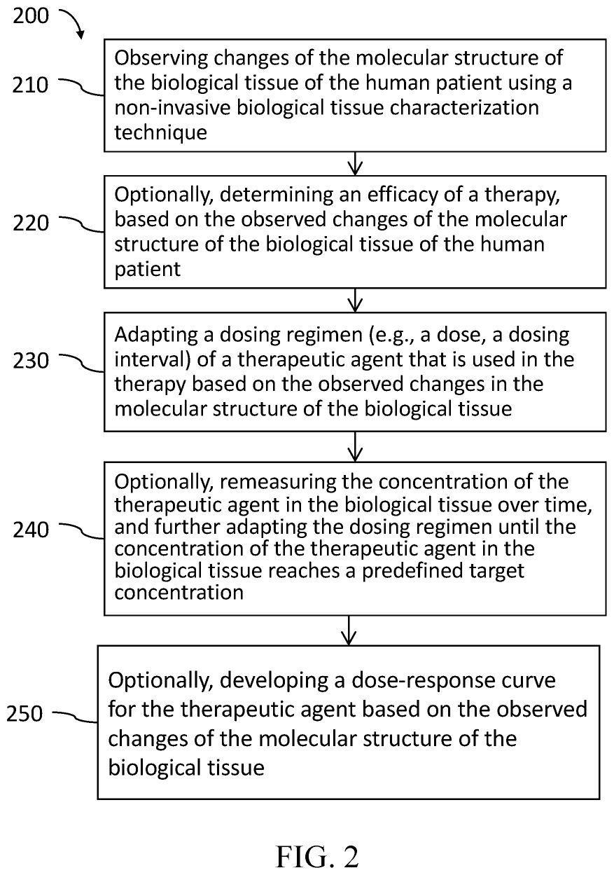

FIG. 2 is a flowchart of an example method 200 for determining an efficacy of a therapy for a disease in a human patient. In block 210, changes of the molecular structure of the biological tissue of the human patient are observed using a non-invasive biological tissue characterization technique. The method performed in block 210 can be similar to that performed in block 120 in method 100 in FIG. 1. In optional block 220, an efficacy of a therapy is determined, based on the observed changes of the molecular structure of the biological tissue of the human patient. The method performed in optional block 220 can be similar to that performed in block 130 in method 100 in FIG. 1. In block 230, a dosing regimen (e.g., a dose, a dosing interval) of a therapeutic agent that is used in the therapy is adapted based on the observed changes in the molecular structure of the biological tissue. In optional block 240, the concentration of the therapeutic agent in the biological tissue is measured (or remeasured) over time, and the dosing regimen is further adapted until the concentration of the therapeutic agent in the biological tissue reaches a predefined target concentration. In optional block 250, a dose-response curve for the therapeutic agent is developed based on the observed changes of the molecular structure of the biological tissue. One or more blocks 210, 220, 230, 240 and 250 of method 200 can be performed wholly or partially by a computer processor (or computer, or server). For example, a computer processor can process data from a characterization technique in block 210 and/or 220.

In some embodiments, the methods disclosed herein include measuring, in the biological tissue, a concentration of a therapeutic agent that is used in the therapy using the non-invasive biological tissue characterization technique. The method can further include adapting a dosing regimen of the therapeutic agent based on the observed changes in the molecular structure of the biological tissue. In some embodiments, the method further includes remeasuring the concentration of the therapeutic agent in the biological tissue over time, and further adapting the dosing regimen (e.g., determining a dosing interval to achieve efficacy or safety) until the concentration of the therapeutic agent in the biological tissue reaches a predefined target concentration (e.g., associated with optimal efficacy and minimal toxicity). In some embodiments, the method further includes developing a therapeutic window for the therapeutic agent, wherein the therapeutic window includes a maximum concentration of the therapeutic agent in the tissue (Cmax), above which there is an increased risk of developing an adverse event, and a minimum concentration of the therapeutic agent in the tissue (Cmin) below which concentrations are ineffective.

In some embodiments, the methods disclosed herein include using a computer workstation to control a characterization device performing the non-invasive biological tissue characterization, such as one or more spectroscopes and/or an X-ray tissue diffractometer. In some cases, the mechanisms and motors of the characterization device, and/or analysis and storage of data from the characterization device (e.g., digital image processing, storing and displaying data received from the two-dimensional pixel detector) can also be performed using the computer workstation. For example, digital image processing can include discrete two-dimensional Fourier transform of images, image segmentation, definition of descriptors of boundaries and regions, and/or recognition of objects within one or more images.

In some embodiments, the methods disclosed herein include controlling one or more characterization devices performing the non-invasive biological tissue characterization technique using a computer workstation. The method can further include digital image processing of one or more images related to the molecular structure of the biological tissue which can also be performed using the computer workstation. For example, the digital image processing can include one or more of: producing a discrete two-dimensional Fourier transform of the one or more images, performing image segmentation of the one or more images, defining descriptors of boundaries or regions in the one or more images, and recognizing objects in the one or more images. The method can further include storing and displaying data received from the characterization device performing the one or more characterization techniques.

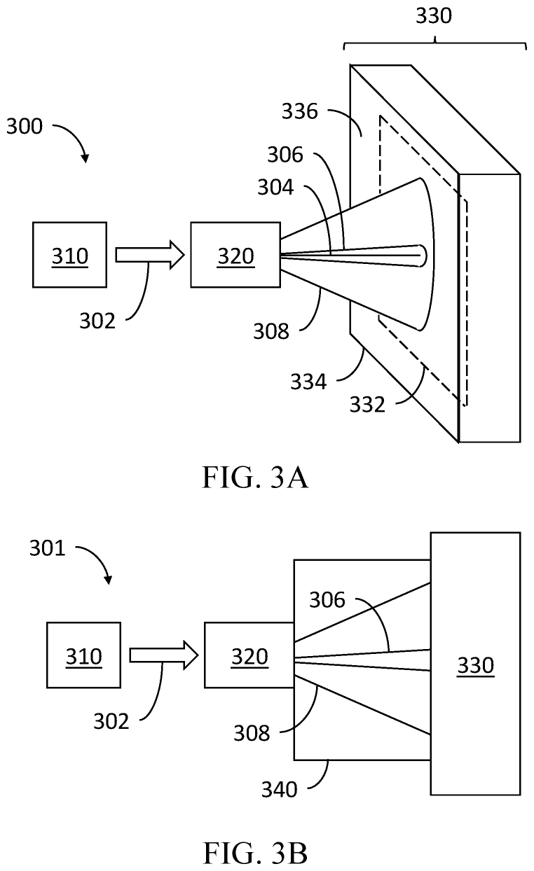

In some embodiments, the non-invasive biological tissue characterization technique of the methods disclosed herein is X-ray diffraction, and the measurements are performed using an X-ray tissue diffractometer. FIGS. 3A and 3B show examples of X-ray tissue diffractometers 300 and 301 that can be used to perform the methods disclosed herein.

FIG. 3A shows a simplified schematic of an example of an X-ray tissue diffractometer 300 including: a positioning area 320 for the biological tissue, an X-ray beam delivery system 310 and a receiver 330. The X-ray beam delivery system 310 provides a primary incident micro-beam of X-rays 302 directed at the biological tissue to be analyzed (the biological tissue being held in positioning area 320). The X-ray beam delivery system 310 can include a radiation source operating in continuous mode, an apparatus forming X-ray micro-beam, at least one monochromator, and at least one collimating and focusing optical device. The receiver 330 can include a two-dimensional pixel detector 332 designed to detect the transmitted micro-beam of X-ray 304 passed through the analyzed biological tissue as well as part or all X-rays 306 and 308 that are diffracted by the biological tissue. X-rays 306 are small-angle x-ray scattering (SAXS) signals, and X-rays 308 are wide-angle x-ray scattering (WAXS) signals, both of which can be detected using receiver 330. In some cases, the two-dimensional pixel detector 332 can be inside a protection container 334 that contains a vacuum (or low pressure) environment or is filled with an inert gas (e.g., neon or helium), and includes a window or wall 336 facing the biological tissue that is substantially transparent to X-rays.

FIG. 3B shows another simplified schematic of an example of an X-ray tissue diffractometer 301, which further comprises a chamber 340 filled with an inert gas (e.g., neon or helium). Chamber 340 is located between the receiver 330 and the positioning area 320 for the biological tissue in a working state (e.g., during the X-ray diffraction characterization of the molecular structure of the biological tissue). In some cases, the chamber 340 can be moveable, and can be moved into a different position in a non-working state. In some cases, receiver 340 can also include two-dimensional pixel detector 332, and chamber 334 with window or wall 336 that is substantially transparent to X-rays.

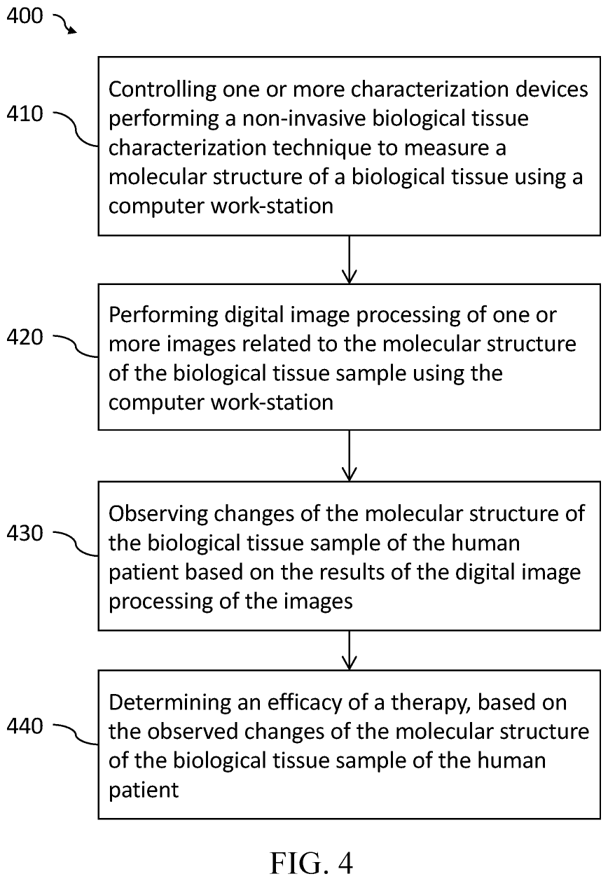

FIG. 4 is a flowchart of an example method 400 for determining an efficacy of a therapy for a disease in a human patient. In block 410, one or more characterization devices performing a non-invasive biological tissue characterization technique are controlled to measure a molecular structure of a biological tissue using a computer workstation (or computer processor, or computer, or server). In block 420, digital image processing of one or more images related to the molecular structure of the biological tissue sample are performed using the computer workstation (or computer processor, or computer, or server). In block 430, changes of the molecular structure of the biological tissue sample of the human patient are observed, based on the results of the digital image processing of the images. In block 440, an efficacy of a therapy is determined, based on the observed changes of the molecular structure of the biological tissue sample of the human patient. Blocks 430 and 440 of method 400 can be performed wholly or partially by a computer workstation (or computer processor, or computer, or server). For example, a computer processor can process data from a characterization technique in block 430 and/or 440.

In some embodiments, the methods disclosed herein include monitoring a clinical response of the patient (e.g., using visualization and diagnostics), such as a general blood test, a biochemical analysis, and/or a urine analysis. In some cases, these clinical responses can provide an indication of the status of the patient using conventional means, which can be used in combination with the methods described herein to further determine the efficacy of a therapy. In some cases, information from the monitoring the clinical response can be used for comparative analysis and testing of the disclosed method, and can be used to further determine the efficacy of the therapy (e.g., in combination with using the non-invasive biological tissue characterization technique to observe changes in the molecular structure of the biological tissue).

In some embodiments, the methods disclosed herein include drawing a dose-response curve for a therapeutic agent used in the therapy, using the observed changes of the molecular structure of the biological tissue. The non-invasive biological tissue characterization techniques of the methods described herein can be used to provide information about the response, which can be used to draw the dose-response curve. In some cases, a dose and/or dosing interval can be determined based on a measurement of a concentration of the therapeutic agent in the tissue (e.g., to achieve efficacy or safety). In some cases, a dose and dosing interval of the therapeutic agent is developed based on the observed change of the molecular structure of the biological tissue (e.g., that is effective while minimizing risk of an adverse event). In some embodiments, a therapeutic window for the therapeutic agent can be developed, which includes a maximum concentration of the therapeutic agent in the tissue (Cmax), above which there is an increased risk of developing the adverse event, and a minimum concentration of the therapeutic agent in the tissue (Cmin) below which concentrations are ineffective.

Conventional methods of determining a therapy efficacy of various diseases in human patients have limitations and disadvantages. For example, the practice of therapeutic drug monitoring (TDM) requires the invasive sampling of blood. Additionally, the blood samples used in TDM are typically analyzed using expensive chromatographic or immunoassay-based machines, for example, in a centralized laboratory. Therefore TDM methods can suffer from long turnaround times, high instrumentation costs and intensive skilled labor requirements. Due to the importance of early detection of a therapy efficacy for treating various diseases and the necessity of quickly changing or modifying a therapy for patients with infectious diseases or cancer, reliable and accurate methods for determining a therapy efficacy various diseases are needed.

The methods disclosed herein include methods for determining an efficacy of a therapy for a disease in a human patient using non-invasive biological tissue characterization. The present methods are advantageous, since determining the efficacy of therapies for various diseases in human patients can be challenging due to the limited pharmacokinetic (PK) and/or pharmacodynamic (PD) information available. The present methods can advantageously address the issues with conventional techniques, using non-invasive biological tissue characterization to directly determine the efficacy of a therapy.

Reference has been made to embodiments of the disclosed invention. Each example has been provided by way of explanation of the present technology, not as a limitation of the present technology. In fact, while the specification has been described in detail with respect to specific embodiments of the invention, it will be appreciated that those skilled in the art, upon attaining an understanding of the foregoing, may readily conceive of alterations to, variations of, and equivalents to these embodiments. For instance, features illustrated or described as part of one embodiment may be used with another embodiment to yield a still further embodiment. Thus, it is intended that the present subject matter covers all such modifications and variations within the scope of the appended claims and their equivalents. These and other modifications and variations to the present invention may be practiced by those of ordinary skill in the art, without departing from the scope of the present invention, which is more particularly set forth in the appended claims. Furthermore, those of ordinary skill in the art will appreciate that the foregoing description is by way of example only, and is not intended to limit the invention.

What is claimed is:

1. A method for determining an efficacy of a therapy for a disease in a human patient, the method comprising:
measuring a molecular structure of a biological tissue of a patient at a first time and at a second time using a non-invasive biological tissue characterization technique;
observing a change of the molecular structure of the biological tissue between the first time and the second time; and
determining the efficacy of the therapy based on the observed change in the molecular structure of the biological tissue, wherein before the first time or between the first time and the second time the patient received the therapy.

2. The method of claim 1, further comprising adapting a dosing regimen of a therapeutic agent that is used in the therapy to characteristics of the human patient, wherein the dosing regimen prevents or corrects both under-dosing deleterious to the efficacy of the therapy and overdosing leading to toxicity.

3. The method of claim 1, wherein a therapeutic agent that is used in the therapy is selected from: anticancer drugs, biologics, antiretrovirals, antiinfectives, psychotropic agents.

4. The method of claim 1, further comprising adjusting the therapy for the patient based on one or more of: a gender of the patient, a metabolic enzyme polymorphism of the patient, a gut microbiota of the patient, a time of administration of a first therapeutic agent that is used in the therapy, a presence of hepatic or renal disease in the patient, and an interaction between the first therapeutic agent and a second therapeutic agent used by the patient.

5. The method of claim 1, wherein the biological tissue is one or more of: a collagen that diffracts x-ray light, a keratin that diffracts x-ray light, and a glycoprotein that diffracts X-ray light.

6. The method of claim 1, wherein the biological tissue comprises one or more of breast tissue, brain tissue, hair, nail, and skin.

7. The method of claim 1, wherein the non-invasive biological tissue characterization technique comprises one or more of: X-ray diffraction, luminescent spectroscopy, selective laser spectroscopy, Raman spectroscopy, spectroscopy in the visible spectral region, and infrared spectroscopy.

8. The method of claim 7, wherein the method further comprises:
measuring, in the biological tissue, a concentration of a therapeutic agent that is used in the therapy using the non-invasive biological tissue characterization technique; and
adapting a dosing regimen of the therapeutic agent based on the observed changes in the molecular structure of the biological tissue.

9. The method of claim 8, further comprising remeasuring the concentration of the therapeutic agent in the biological tissue over time, and further adapting the dosing regimen until the concentration of the therapeutic agent in the biological tissue reaches a predefined target concentration.

10. The method of claim 8, further comprising developing a therapeutic window for the therapeutic agent, wherein the therapeutic window includes a maximum concentration of the therapeutic agent in the tissue (Cmax), above which there is an increased risk of developing an adverse event, and a minimum concentration of the therapeutic agent in the tissue (Cmin) below which concentrations are ineffective.

11. The method of claim 7, further comprising:
controlling one or more characterization devices performing the non-invasive biological tissue characterization technique using a computer workstation;
performing digital image processing of one or more images related to the molecular structure of the biological tissue using the computer workstation; and
storing and displaying data received from the characterization device performing the one or more characterization techniques.

12. The method of claim 11, wherein the digital image processing comprises one or more of: producing a discrete two-dimensional Fourier transform of the one or more images, performing image segmentation of the one or more images, defining descriptors of boundaries or regions in the one or more images, and recognizing objects in the one or more images.

13. The method of claim 7, wherein the X-ray diffraction uses an X-ray tissue diffractometer comprising:
a positioning area for the biological tissue;
an X-ray beam delivery system providing a primary incident micro-beam of X-rays directed at the biological tissue to be analyzed, wherein the X-ray beam delivery system comprises:
a radiation source operating in a continuous mode;
an apparatus forming the primary incident micro-beam of X-rays;
a monochromator; and
at least one of a collimating optical device and a focusing optical device; and
a receiver comprising a two-dimensional pixel detector designed to detect a transmitted micro-beam of X-rays passed through the analyzed biological tissue as well as part or all of X-rays that are diffracted by the biological tissue.

14. The method of claim 13, wherein the two-dimensional pixel detector is inside a protection container, wherein the protection container comprises a vacuum or an inert gas environment, and a window or wall facing the tissue that is substantially transparent to the X-rays.

15. The method of claim 14, wherein the inert gas is neon or helium.

16. The method of claim 13, wherein the X-ray tissue diffractometer further comprises a chamber filled with an inert gas wherein the chamber is located between the two-dimensional pixel detector and the tissue during the X-ray diffraction characterization of the molecular structure of the biological tissue.

17. The method of claim 16, wherein the inert gas is neon or helium.

18. The method of claim 1, wherein the disease in the human patient comprises diseases of the immune system, rheumatic diseases, cancer, or diseases of one or more of the: skin, stomach, liver, rectum, colon, esophagus, pancreas, bladder, vagina, lung, oropharynx, nasopharynx, oral mucosa, tongue, brain, thyroid, prostate, breast, cervix, ovary, urological organs, endocrine organs, veins, lymph nodes, mammary glands, respiratory organs, digestive organs, heart, blood vessels, colon, ear, throat, or nose.

19. The method of claim 1, further comprising monitoring a clinical response of the patient comprising one or more of: a general blood test, a biochemical analysis, and a urine analysis, and using information from the monitoring to further determine the efficacy of the therapy.

20. The method of claim 1, further comprising determining a dose-response curve for a therapeutic agent used in the therapy, using the observed changes of the molecular structure of the biological tissue.

21. The method of claim 1, further comprising developing a dose and dosing interval for a therapeutic agent used in the therapy using the observed changes of the molecular structure of the biological tissue.

22. The method of claim 1, further comprising measuring the molecular structure of the biological tissue at a plurality of times using the non-invasive biological tissue characterization technique, wherein the plurality of times comprises the first time and the second time.

23. The method of claim 22, wherein the observing the change of the molecular structure of the biological tissue further comprises comparing regression coefficients of functions fit to data from the measurements of the molecular structure of the biological tissue at the plurality of times.
